SEQUENCE LISTING (14 sequences)

SEQ ID NO: 1 - protein, 16 aa, homo sapiens
APSNEAWDNLDSDIRR

SEQ ID NO: 2 - protein, 836 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 3 - protein, 779 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 4 - protein, 781 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 5 - protein, 751 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEDTPVRKLQANKKVQGSRRRLREGRSQ

SEQ ID NO: 6 - protein, 809 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 7 - protein, 749 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQEEVTKVTKFIEGGDGHLFEDEEIKRLLQGDTPVRKLQANKKVQGSRRRLREGRSQ

SEQ ID NO: 8 - protein, 721 aa, homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQEDTPVRKLQANKKVQGSRRRLREGRSQ

SEQ ID NO: 9 - protein, 838 aa, mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYTTKIITKVVEPKIKVIQGSLQPIIKTEGPAMTKIQIEGDPDFRLIKEGETVTEVIHGEPVIKKYTKIIDGVPVEITEKQTREERIITGPEIKYTRISTGGGETGETLQKFLQKEVSKVTKFIEGGDGHLFEDEEIKRLLQGDTPAKKIPANKRVQGPRRRSREGRSQ

SEQ ID NO: 10 - protein, 811 aa, mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYRPAMTKIQIEGDPDFRLIKEGETVTEVIHGEPVIKKYTKIIDGVPVEITEKQTREERIITGPEIKYTRISTGGGETGETLQKFLQKEVSKVTKFIEGGDGHLFEDEEIKRLLQGDTPAKKIPANKRVQGPRRRSREGRSQ

SEQ ID NO: 11 - protein, 810 aa, mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYTTKIITKVVEPKIKVIQGSLQPIIKTEGPAMTKIQIEGDPDFRLIKEGETVTEVIHGEPVIKKYTKIIDGVPVEITEKQTREERIITGPEIKYTRISTGGGETGETLQKFLQKDTPAKKIPANKRVQGPRRRSREGRSQ

SEQ ID NO: 12 - protein, 784 aa, mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYTTKIITKVVEPKIKVIQGSLQPIIKTEGPAMTKIQIEGDPDFRLIKEGETVTEVIHGEPVIKKYTKIIDGVPVEITEKQTREERIITDTPAKKIPANKRVQGPRRRSREGRSQ

SEQ ID NO: 13 - protein, 783 aa, mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYRPAMTKIQIEGDPDFRLIKEGETVTEVIHGEPVIKKYTKIIDGVPVEITEKQTREERIITGPEIKYTRISTGGGETGETLQKFLQKDTPAKKIPANKRVQGPRRRSREGRSQ

SEQ ID NO: 14 - protein, 16 aa, mus musculus
APSNEAWENLDSDIRR

(19) United States
(12) Patent Application Publication
(10) Pub. No.: US 2024/0141026 A1
(43) Pub. Date: May 2, 2024

(54) USE OF A PERIOSTIN ANTIBODY FOR TREATING INFLAMMATION, FIBROSIS AND LUNG DISEASES

(71) Applicants: Centre National de la Recherche Scientifique, PARIS (FR); Université d'Orléans, ORLEANS CEDEX 2 (FR)

(72) Inventors: Magdiel PEREZ-CRUZ, ORLEANS (FR); Bernhard RYFFEL, SAINT-DENIS-EN-VAL (FR)

(21) Appl. No.: 18/548,850

(22) PCT Filed: Mar. 4, 2022
(86) PCT No.: PCT/EP2022/055605

§ 371 Date: Sep. 1, 2023

(30) Foreign Application Priority Data
Mar. 4, 2021 (EP) 21305260.8

(51) Int. Cl. C07K 16/18; A61P 11/06; A61P 29/00; A61K 39/00

(52) U.S. Cl. CPC C07K 16/18; A61P 11/06; A61P 29/00; A61K 2039/505; C07K 2317/24; C07K 2317/92

(57) ABSTRACT

The present invention is based on the surprising finding that the MPC5B4 anti-periostin monoclonal antibody can effectively prevent and treat inflammation, fibrosis and worsening of pulmonary inflammation and respiratory diseases in various animal models. The MPC5B4 anti-periostin monoclonal antibody recognizes specifically the peptide sequence of SEQ ID NO:1, corresponding to the amino acid sequence 136-151 located within the fasciclin (FAS)1-1 domain of the periostin protein (POSTN), inhibiting its binding to the αvβ3 integrin.

### CROSS-REFERENCE TO RELATED APPLICATIONS

The present application is a filing under 35 U.S.C. 371 as the National Stage of International Application No. PCT/EP2022/055605, filed Mar. 4, 2022, entitled “USE OF A PERIOSTIN ANTIBODY FOR TREATING INFLAMMATION, FIBROSIS AND LUNG DISEASES,” which claims priority to European Application No. 21305260.8 filed with the Intellectual Property Office of Europe on Mar. 4, 2021, both of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4692-12600 BNT241857USPC Sequence listing; created on Aug. 31, 2023; and having a file size of 81 KB.

The information in the Sequence Listing is incorporated herein in its entirety for all purposes.

### SUMMARY OF THE INVENTION

The present invention is based on the surprising finding that the MPC5B4 anti-periostin monoclonal antibody can effectively prevent and treat inflammation, fibrosis and worsening of pulmonary inflammation and respiratory diseases in various animal models. The MPC5B4 anti-periostin monoclonal antibody recognizes specifically the peptide sequence of SEQ ID NO:1, corresponding to the amino acid sequence 136-151 located within the fasciclin (FAS)1-1 domain of the periostin protein (POSTN), inhibiting its binding to the αvβ3 integrin.

### BACKGROUND OF THE INVENTION

Inflammation is the body's natural defense mechanism to remove harmful stimuli such as pathogens, irritants and damaged cells and initiate the healing process. In general, inflammation is classified as acute or chronic inflammation. Acute inflammation is originally a beneficial process that helps to immobilize the injured region and activates the immune system to heal injuries, although it may lead to acute inflammation such as Acute Respiratory Distress Syndrome (ARDS), a main cause of mortality in COVID-19 for instance. Chronic inflammation, on the other hand, turns into a problem as it may destroy healthy tissues in a misdirected attempt at initiating the healing process¹.

Airway inflammation is usually caused by pathogens or by exposure to toxins, pollutants, irritants, and allergens. TLRs recognize molecular patterns shared by pathogens and activate inflammatory cells via pathways such as nuclear factor kappa-light-chain-enhancer of activated B-cells (NF-κB) to produce growth factors, chemokines, pro-inflammatory cytokines such as interleukin 8 (IL-8) and tumor necrosis factor alpha (TNF-α). IL-8 evokes neutrophils recruitment and TNF-α induces expression of endothelial cell adhesion molecules in lung capillaries. Moreover, many of the known inflammatory target proteins, such as matrix metalloproteinase-9 (MMP-9), intercellular adhesion molecule-1 (ICAM-1), vascular cell adhesion molecule-1 (VCAM-1), cyclooxygenase-2 (COX-2), and cytosolic phospholipase A2 (cPLA2), are specifically associated with airways inflammation in response to various stimuli¹.

The lung is a vital organ for providing mandatory oxygen for all organs in the body, and excessive inflammation can be life threatening. A delicate balance between inflammation and anti-inflammation is essential for lung homeostasis.

According to the World Health Organization, the prevalence of lung and airway inflammatory diseases has considerably increased in the last thirty years, concerning millions of patients worldwide. Among these important diseases, one can cite asthma and chronic obstructive pulmonary disease (COPD), that affect more than 500 million people worldwide.

The current consensus definition of asthma is “an heterogeneous disease, usually characterized by chronic airway inflammation, which is defined by the history of respiratory symptoms such as wheeze, shortness of breath, chest tightness and cough that vary over time and in intensity, together with variable airflow obstruction”. Patients with asthma have variable airflow obstruction and airway hyper-responsiveness (AHR). Asthma affects 10%-12% of the adult population in Europe. Patients with severe disease do not respond well to conventional anti-inflammatory corticosteroids that is the mainstay treatment of mild-moderate asthma².

On the other hand, COPD is characterized by persistent airflow limitation that is usually progressive and associated with an enhanced chronic inflammatory response in the airways and the lung to noxious particles or gases. Exacerbations and comorbidities contribute to the overall severity in individual patients. COPD is expected to rise from the 4th to the 3rd leading cause of morbidity and mortality worldwide within the next 5 years. According to the World Health Organization, approximately 3 million people in the world die as a consequence of COPD every year. In developed countries, the leading risk factor for COPD is cigarette smoking with smokers constituting more than 90% of COPD patients. In less-well developed countries, biomass fuel used in cooking and other environmental pollutants are major factors. The pathologic features of COPD are lung parenchymal destruction (pulmonary emphysema), inflammation of the small (peripheral) airways (respiratory bronchiolitis), and inflammation of the central airways. Inflammation occurs within all these compartments (central and peripheral airways and lung parenchyma). The major sites of airflow obstruction are the small airways and lung parenchyma in COPD².

Idiopathic pulmonary fibrosis (IPF) is a type of chronic progressive fibrotic lung disease characterized by a rapid and irreversible decline in lung function. The tissue in the lungs becomes rigid, which affects the tissue that surrounds the air/alveoli in the lungs. Symptoms typically include gradual onset of shortness of breath and a dry cough. Complications may include pulmonary hypertension, heart failure, pneumonia, or pulmonary embolism. The cause is unknown. Risk factors include cigarette smoking, certain viral infections, and a family history of the condition. The underlying mechanism involves progressive fibrosis of the lungs. About 5 million people are affected globally. Average life expectancy following diagnosis is about four years.

Acute respiratory distress syndrome (ARDS) is a life-threatening condition of seriously ill patients, characterized by poor oxygenation, pulmonary infiltrates, and acuity of onset. On a microscopic level, the disorder is associated with capillary endothelial injury and diffuse alveolar damage. ARDS is defined as an acute disorder that starts within 7 days of the inciting event and is characterized by bilateral lung infiltrates and severe progressive hypoxemia in the absence of any evidence of cardiogenic pulmonary edema. ARDS is defined by the patient's oxygen in arterial blood (PaO₂) to the fraction of the oxygen in the inspired air (FiO₂). These patients have a PaO₂/FiO₂ ratio of less than 300. Once ARDS develops, patients usually have varying degrees of pulmonary artery vasoconstriction and, subsequently, may develop pulmonary hypertension. ARDS carries a high mortality, and few effective therapeutic modalities exist to combat this condition³.

In common with most chronic inflammatory diseases, corticosteroids are able to attenuate inflammation in asthmatic airways leading to a reversal in the decline in forced expiratory volume in one second (FEV1) associated with the disease and a reduction in AHR back to normal levels. However, corticosteroids do not cure asthmatics as many of the symptoms of asthma and the inflammation return upon their discontinuation. Moreover, severe asthmatics poorly respond to corticosteroids, often have more frequent exacerbations requiring hospitalization and depression associated with the chronic nature of the disease, which is not controlled by conventional therapy. In COPD patients, corticosteroid treatment is associated with adverse effects such as increased risk of oropharyngeal candidiasis, hoarseness, and pneumonia.

Other treatments involve targeting inflammatory mediators that have been implicated in lung inflammation, including multiple cytokines 4, chemokines, and growth factors such as TGF-β⁵. In view of the complexity of the inflammatory signalling, blocking a single mediator is unlikely to be very effective in these complex diseases, and mediator antagonists have so far not proved to be very effective compared with drugs that have a broad spectrum of anti-inflammatory effects, such as corticosteroids. For example, the lack of effect of anti-TNFα in COPD is in stark contrast to the beneficial effect seen in other chronic inflammatory diseases such as rheumatoid arthritis.

In addition, using unspecific immunosuppressive treatments often induces side-effects because it lowers the immune defenses of the patients and render them sensitive to virus or bacterial infection.

In this context, there is an urgent need for a new effective drug that could prevent, treat or even ultimately cure these patients as they will have a profound effect on an individual patient's welfare and also on the huge associated societal costs.

### DESCRIPTION OF THE INVENTION

The present inventors herein demonstrate the beneficial effect on inflammatory lung diseases of a monoclonal antibody which is highly specific of a particular domain on the periostin protein (POSTN) and can inhibit the binding of POSTN to one of its target, namely to integrins αvβ3, and therefore the migration of the inflammatory cells. Their results show that said antibody reduces inflammation, lung fibrosis and exacerbation in several animal models (cf. FIGS. 2-6 and 8). Its high specificity and strong inhibitory activity specifically on POSTN binding to αvβ3 is thought to explain its high therapeutical potential for blocking the deleterious effects of POSTN in lung inflammation and fibrosis. By blocking the signaling events resulting from the binding of periostin to αvβ3 only, the antibody of the invention advantageously prevents and treats lung inflammation, but does not affect the overall steady-state immune response. This proposed treatment will therefore not induce side-effects that are observed by using unspecific immunosuppressive treatments. It will also be more beneficial than the novel therapies targeting particular cytokines⁴ or involving anti-IgE neutralizing therapies, that also enhance the risk of infection.

In a first aspect, the present invention relates to the use of a monoclonal anti-periostin (POSTN) IgG antibody that recognizes specifically and with high affinity the peptide sequence of SEQ ID NO:1 (APSNEAWDNLDSDIRR) in the fasciclin (FAS)1-1 domain of periostin, or an antigen-binding fragment thereof, as a medicament, preferably for preventing and/or treating a pulmonary, inflammatory or respiratory disease.

It is noteworthy that the OC-20 antibodies (IgM) disclosed by Orecchia et al, 2011¹⁵ bind to the Fas 1-2 domain of POSTN and not to the (FAS)1-1 domain of periostin. Moreover, the OC-20 antibodies have an inhibitory activity on both αvβ3 and αvβ5 integrins, not only on αvβ3 integrins. Although this antibody has an effect on reducing lung fibrosis²³ and on inflammatory responses to aeroallergens in mice models²⁴, its therapeutic use on human pulmonary fibrosis has however never been proposed nor suggested. More importantly, none of these studies proposed to use an antibody as a therapeutic drug in humans. This can be due to the fact that the tested antibody was an IgM (and therefore is capable of activating the complement) that recognized mouse POSTN and not human POSTN.

Antibody of the Invention

The term “antibody” is used herein in the broadest sense and specifically covers monoclonal antibodies (including full length monoclonal antibodies) of any isotype such as IgG, IgM, IgA, IgD, and IgE, polyclonal antibodies, multispecific antibodies, chimeric antibodies, and antigen-binding fragments.

In a preferred embodiment, the antibody of the invention is a monoclonal IgG, which elicits a more robust immune response as compared to other types of immunoglobulins. In a more preferred embodiment, the antibody of the invention is a monoclonal IgG1, i.e., it contains two γ1 heavy chains. The IgG1 isotype is not only the most abundant isotype found in human serum, but it is also the best studied and best-understood human antibody form. Additionally, the IgG1 activity profile reasonably suits soluble targets such as POSTN.

As used herein, the term “monoclonal antibody” refers to an antibody arising from a nearly homogeneous antibody population. More particularly, the individual antibodies of a population are identical except for a few possible naturally-occurring mutations which can be found in minimal proportions. In other words, a monoclonal antibody consists of a homogeneous antibody population arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is generally characterized by heavy chains of one and only one class and subclass, and light chains of only one type. Monoclonal antibodies are highly specific and are directed against a single epitope on an antigen, said epitope being for example the peptide of SEQ ID NO:1. The sequence of this epitope, formed by contiguous amino acids, is typically retained upon exposure to denaturing agents.

The epitope of SEQ ID NO:1 (APSNEAWDNLDSDIRR) corresponds to the amino acid sequence 136-151 located within the fasciclin (FAS)1-1 domain of the human periostin protein (POSTN). This highly conserved domain is also present in the mouse periostin protein, with a single conservative point mutation D-E in position 145 (APSNEAWENLDSDIRR, designated as SEQ ID NO:14), also recognized as epitope by the said monoclonal antibodies.

The human POSTN protein has 7 isoforms that are described in UniProt under the following numbers:
Q15063 (isoform 1, human): SEQ ID NO:2
Q15063-2 (isoform 2, human): SEQ ID NO:3
Q15063-3 (isoform 3, human): SEQ ID NO:4
Q15063-4 (isoform 4, human): SEQ ID NO:5
Q15063-5 (isoform 5, human): SEQ ID NO:6
Q15063-6 (isoform 6, human): SEQ ID NO:7
Q15063-7 (isoform 7, human): SEQ ID NO:8

All of them contain SEQ ID NO:1.

The mouse POSTN protein has 5 isoforms that are described in UniProt, under the following numbers:
Q62009 (isoform 1, mouse): SEQ ID NO:9
Q62009-2 (isoform 2, mouse): SEQ ID NO:10
Q62009-3 (isoform 3, mouse): SEQ ID NO:11
Q62009-4 (isoform 4, mouse): SEQ ID NO:12
Q62009-5 (isoform 5, mouse): SEQ ID NO:13

All of them contain SEQ ID NO:1 with a single conservative point mutation D-E in the position 145.

Periostin (POSTN) is a member of the matricellular family of proteins. Matricellular proteins are defined by their ability to bind both to the extracellular matrix (ECM) and to cell surface receptors. POSTN is known to play a role in airway development and alveolar epithelial repair and is notably up-regulated in infants with bronchopulmonary dysplasia⁶. POSTN influences cellular interactions with integrin receptors and influences production and localization of fibrogenic cytokines and growth factors^{7, 8}. POSTN facilitates tissue remodeling and collagen crosslinking through its interaction with other ECM proteins and enzymes⁹. It is expressed in several different human tissues including the lung. POSTN is considered to be a key factor in the evolution of inflammation and interstitial fibrosis and implicated in the pathogenesis of several chronic lung diseases including asthma and interstitial lung disease (ILD)⁷.

Periostin (POSTN) was originally identified as osteoblast-specific factor 2 in a mouse osteoblast cell line¹⁰ and is expressed in the periosteum and in the periodontal ligament. That's why it has been also named as “OSF-2”, “OSF2”, and “PDLPN”.

The POSTN molecule is composed of a cysteine-rich domain within the N-terminal region, four fasciclin I domains, and an alternative splicing domain within the C-terminal region. Interestingly, up to nine splice variants have been identified, but the full-length transcript encodes a 93.3 kDa secreted protein that includes all exons 11 POSTN binds type I collagen and fibronectin and has been shown to be involved in collagen fibrillogenesis⁸. POSTN binds integrins αvβ3, αvβ5 and α6β4 leading to activation of the Akt/Protein kinase B (PKB) and Focal adhesion kinase (FAK) signalling pathways and promotes cell survival, angiogenesis and resistance to hypoxia-induced apoptosis¹². It also binds to a number of ECM proteins, dependent upon the c-terminus, such as heparin, fibronectin, collagen V and tenascin C, thereby enhancing tumour cell invasiveness by affecting ECM fibrillogenesis⁸ and metastasis of various tumors¹³.

In a preferred embodiment, the monoclonal antibody of the invention or antigen-binding fragments thereof is able to inhibit the binding of all isoforms of the human and mouse protein POSTN to the αvβ3 integrins expressed by epithelial cells. In other terms, the invention provides antagonistic antibodies or antigen-binding fragments thereof capable of inhibiting the interaction of the all isoforms of the human and mouse POSTN proteins with the αvβ3 integrins expressed by epithelial cells.

The inhibitory activity of the antibody of the invention or antigen-binding fragments thereof can be assessed by evaluating its influence on the binding of human or mouse POSTN to αvβ3 and/or αvβ5-coated ELISA plates as disclosed in Field et al, 2016¹⁴. An inhibitory activity can be concluded if the IC₅₀ for inhibiting POSTN binding is below 30 μg/mL in the conditions of FIG. 2a of Field et al¹⁴.

The inhibitory activity of the antibody of the invention can also be documented by studying its influence on the migration of migrating cells such as ECFC, in response to recombinant human or mouse POSTN or its Fas1-1 domain. As disclosed in Field et al¹⁴ (FIG. 5), the antibodies of the invention are preferably able to reduce POSTN-induced migration of ECFCs to the level of negative controls.

The high specificity and strong inhibitory activity of the antibody of the invention or antigen-binding fragments thereof is thought to explain the strong effect observed in murine models of human lung disease, as shown in the example part below.

As a matter of fact, the antibodies of the present application have a high affinity for SEQ ID NO:1. More preferably, they possess a very low dissociation constant with SEQ ID NO:1. For example, this very low dissociation constant is inferior or equal to 50 nM and may reach down to the picomolar range (10⁻¹² M). More specifically, the antibodies of the invention or functional antigen-binding fragments thereof have a dissociation constant (K_{D}) with SEQ ID NO:1 of about 0.08.

As used herein, the term “K_{D}” refers to the dissociation constant of a particular antibody/antigen interaction. As used herein the term “binding affinity” or “affinity of binding” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Competition experiments with competing peptides such as those disclosed in Field et al¹⁴, can be also performed.

A titration of the anti-POSTN antibody (MPC5B4) was made on periostin-coated plates. This does not allow for real affinity calculation but gives an idea of the avidity. The 50% max binding was calculated, for human POSTN (0.0063 nM, Kd=0.08) and mouse POSTN (0.00419 nM).

In a preferred embodiment, the antibody of the invention, or functional fragment thereof, does not bind to any other epitope in human POSTN protein (because it has a dissociation constant superior to 1 mM with all the other epitopes). Specifically, it does not bind to the Fas 1-2 domain of POSTN, as do the OC-20 antibodies (IgM) disclosed by Orecchia et al, 2011¹⁵.

Obtention of the Antibody of the Invention

In a preferred embodiment, the antibody of the invention can be the IgG1 monoclonal antibody called “MPC5B4” disclosed by Field et al, 2016¹⁴, which is available upon request at the Ludwig Institute for Cancer Research (Avenue Hippocrate 74, Box B1.74.03 B-1200 Brussels, BELGIUM).

As disclosed in Field et al, 2016¹⁴, other efficient monoclonal antibodies can be produced by immunizing mice with the epitope (SEQ ID NO:1) or the whole POSTN protein having a sequence chosen in the group consisting of SEQ ID NO:2 to 13, conjugated to an immunogenic carrier such as ovalbumine (OVA). This conjugation can be performed by any conventional means. An example of said conjugation is given in Field et al¹⁴, where full length POSTN was first polymerized for 1 hr with 100 mM N-(3-dimethylamino propyl)-N′-ethylcarbodiimide in the presence of 10 mM N-hydroxysulfosuccinimide in 0.1M MES buffer pH4.8. After dialysis against 0.1M acetate buffer pH 5.8, polymerized POSTN was conjugated to glutaraldehyde activated OVA. More details are given in Uyttenhove C et al, 2011¹⁶.

It is preferred, as suggested in Field et al¹⁴ to produce the antibodies in mice whose immune system has been powerfully stimulated, e.g., by infecting same with a lactate dehydrogenase-elevating virus (LDV) virus, a single stranded positive sense RNA enveloped arterivirus with B-cell activation properties, by intraperitoneal injection of infected plasma¹⁷.

Alternatively, one can use POSTN-deficient mice, that have no endogenous POSTN protein (i.e., no isoform of the POSTN protein), as reported in the prior art (see the papers cited in the discussion of Field et al¹⁴).

Field et al. suggests that such a vaccination procedure breaches the immune self-tolerance of the mice and enables to generate several antibodies that recognize specifically the SEQ ID NO:1 of the human and mouse POSTN protein of SEQ ID NO:2-13, without cross-reacting with TGFβ1.

These mice can be immunized in the footpads with 10 μg POSTN (any isoform of SEQ ID NO:2-13) or SEQ ID NO:1—conjugated to ovalbumin (OVA), preferably several times at weekly intervals, e.g., three times at 2 weeks intervals. An intravenous injection of 5 μg POSTN (any isoform of SEQ ID NO:2-13 or the epitope of SEQ ID NO:1) can be done 4 days before cell fusion with the myeloma cells.

These immunized mice will produce lymphocytes that generate antibodies that specifically binds to the SEQ ID NO:1 epitope in POSTN protein. These lymphocytes are collected and then fused with myeloma cells using a suitable fusing agent, such as polyethylene glycol, to form a hybridoma cell. The hybridoma cells thus prepared can be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, California USA, and SP-2 or X63-Ag8-653 cells available from the American Type Culture Collection, Rockville, Maryland USA.

Culture medium in which hybridoma cells are growing is assayed for production of monoclonal antibodies directed against the antigen. Preferably, the binding specificity of monoclonal antibodies produced by hybridoma cells is determined by an in vitro binding assay, such as radioimmunoassay (RIA) or enzyme-linked immunoabsorbent assay (ELISA).

After hybridoma cells that produce antibodies of the desired specificity, affinity, and/or activity, are identified, the clones may be subcloned by limiting dilution procedures and grown by standard methods. Suitable culture media for this purpose include, for example, D-MEM or RPMI-1640 medium.

The monoclonal antibodies secreted by the subclones are suitably separated from the culture medium, ascites fluid, or serum by conventional immunoglobulin purification procedures such as, for example, protein G- or protein A-Sepharose, hydroxylapatite chromatography, gel electrophoresis, dialysis, or affinity chromatography.

In a further embodiment, antibodies or antibody fragments can be isolated from antibody phage libraries generated using the techniques described in McCafferty et al., Nature, 348: 552-554 (1990). Clackson et al., Nature, 352: 624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et al., Biotechnology, 10: 779-783 (1992)), as well as combinatorial infection and in vivo recombination as a strategy for constructing very large phage libraries (Waterhouse et al., Nuc. Acid. Res., 21: 2265-2266 (1993)). Thus, these techniques are viable alternatives to traditional monoclonal antibody hybridoma techniques for isolation of monoclonal antibodies.

The specificity/avidity of the antibodies of the invention can for example be assessed by ELISA experiments, using microplates coated with the polypeptide of SEQ ID NO:1, human POSTN (any isoform), mouse POSTN (any isoform) or other related proteins. The selection of the antibodies of interest will be performed on the basis of their relative avidity/specificity.

Mutations in the Fc Domain

It must be understood here that the invention preferably does not relate to antibodies in natural form, i.e., they are not taken from their natural environment but are isolated or obtained by purification from natural sources, or obtained by genetic recombination or chemical synthesis. Thus, they can carry “unnatural” amino acids as will be described below.

For example, it is possible to increase the half-life of the antibodies or fragments of the invention by introducing the following amino acid mutations:
M252Y/S254T/T256E (“YTE”): this mutation increases the binding of the IgG or Fc domains to the IgG recycling receptor, FcRn, leading to a prolonged half-life¹⁸.
M428L/N434S (“LS”): this mutation increases the binding of the IgG or Fc domains to the IgG recycling receptor, FcRn, leading to a prolonged half-life¹⁹.
L309D/Q311H/N434S (“DHS”): this mutation increases the binding of the IgG or Fc domains to the IgG recycling receptor, FcRn, leading to a prolonged half-life²⁰
T307A/E380A/N434A: this mutation increases the binding of the IgG or Fc domains to the IgG recycling receptor, FcRn, leading to a prolonged half-life²¹.

Moreover, as the antibodies and fragments of the invention are intended to be used in the treatment and/or therapy in humans, their potential immunogenicity and deleterious effects should be minimized by any means.

It is therefore recommended to modify the Fc regions of these antibodies in order to modify their effector functions, as already proposed in the art. In particular, it is better to mutate the Fc region of the antibodies in order to avoid the activation not only of the receptors FcγR (FcγRI, FcγRII, FcγRIII, FcγRIIIA, FcγRIIIB, Fcγn) but also of the C1q component of the complement, which plays important roles in opsonization, lysis of cell pathogens, and inflammatory responses.

As used herein, the term “Fc region” is used to define a C-terminal region of an IgG heavy chain. Although the boundaries may vary slightly, the human IgG heavy chain Fc region is defined to stretch from Cys226 to the carboxy terminus. The Fc region of an IgG comprises two constant domains, CH2 and CH3. The CH2 domain of a human IgG Fc region usually extends from amino acids 231 to amino acid 341. The CH3 domain of a human IgG Fc region usually extends from amino acids 342 to 447. The CH2 domain of a human IgG Fc region usually extends from amino acid 231-340. The CH2 domain is unique in that it is not closely paired with another domain. Rather, two N-linked branched carbohydrate chains are interposed between the two CH2 domains of an intact native IgG.

As used herein, an Fc region that “lacks effector function” does not bind the Fc receptor and/or does not bind the C1q component of complement nor trigger the biological responses characteristic of such binding.

It is possible to impair the effector function of antibodies by generating Fc regions that are not glycosylated (or “aglycosylated”) at its usual glycosylation sites.

The term “glycosylation site” refers to an amino acid residue that is recognized by a mammalian cell as a location for the attachment of sugar residues. Amino acid residues to which carbohydrates, such as oligosaccharides, are attached are usually asparagine (N-linkage), serine (O-linkage), and threonine (O-linkage) residues. The specific sites of attachment usually have a characteristic sequence of amino acids, referred to as a “glycosylation site sequence.” The glycosylation site sequence for N-linked glycosylation is: -Asn-X-Ser- or -Asn-X-Thr-, where X can be any of the conventional amino acids, other than proline. The Fc region of human IgG has two glycosylation sites, one in each of the CH2 domains. The glycosylation that occurs at the glycosylation site in the CH2 domain of human IgG is N-linked glycosylation at the asparagine at position 297 (Asn 297).

In particular, it is possible to modify the Fc regions of the antibodies of the invention by mutating them with any of the following mutations:
N297A: this mutation replaces the asparagine able to receive N-glycosylation. This N-glycosylation is necessary for the interaction between the Fc region of IgG and human low-affinity FcγR (CD32A, CD32B, CD32C, CD16A, CD16B). It does not affect the interaction of IgG with the high-affinity FcγR, FcγRI/CD64.
N297D: similar mutation to N297A with same consequences on FcγR binding.
L234A, L235A (LALA): this double mutation abolishes the interaction between the Fc region of IgG and human low-affinity FcγR (CD32A, CD32B, CD32C, CD16A, CD16B). It does not affect the interaction of IgG with the high-affinity FcγR, FcγRI/CD64.
L234A, L235A, P329G (LALAPG): this triple mutation abolishes the interaction between the Fc region of IgG and all human FcγR, whether low-affinity FcγR (CD32A, CD32B, CD32C, CD16A, CD16B) or high-affinity FcγR (CD64).

Any of this mutation can be used to generate an efficient therapeutic antibody that can be safely administered to human beings.

Moreover, all the mutations known in the art to enhance the efficiency and reduce the adverse side effects of therapeutic antibodies are herewith encompassed.

Humanization of the Antibody

In another aspect, the invention relates to chimeric or humanized antibodies, or antigen-binding fragments, which can be obtained by genetic engineering or by chemical synthesis. Specifically, the POSTN antibodies of the invention are chimeric antibodies.

The term “chimeric antibody” as used herein refers to an antibody containing a natural variable region (light chain and heavy chain) derived from an antibody of a given species in combination with constant regions of the light chain and of the heavy chain of an antibody of a species heterologous to said given species. Thus, a “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass. Such chimeric antibodies, or fragments of same, can be prepared by recombinant engineering. For example, the chimeric antibody could be produced by cloning recombinant DNA containing a promoter and a sequence coding for the variable region of a non-human monoclonal antibody of the invention, notably murine, and a sequence coding for the human antibody constant region. A chimeric antibody according to the invention coded by one such recombinant gene could be, for example, a mouse-human chimera, the specificity of this antibody being determined by the variable region derived from the murine DNA and its isotype determined by the constant region derived from human DNA. In another aspect, the present invention provides humanized antibodies, or antigen-binding fragments thereof.

As used herein, the term “humanized antibody” refers to a chimeric antibody which contain minimal sequence derived from non-human immunoglobulin. In one embodiment, a humanized antibody is a human immunoglobulin (recipient antibody) in which residues from a CDR of the recipient are replaced by residues from a CDR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the Heavy (H) chain, and in the Light (L) chain, no more than 3. The humanized antibody optionally will also comprise at least a portion of an immunoglobulin constant region (Fc), typically that of a human immunoglobulin.

The goal of humanization is a reduction in the immunogenicity of a xenogenic antibody, such as a murine antibody, for introduction into a human, while maintaining the full antigen binding affinity and specificity of the antibody. Humanized antibodies, or antibodies adapted for non-rejection by other mammals, may be produced using several technologies such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host.

Strategies and methods for the resurfacing of antibodies, and other methods for reducing immunogenicity of antibodies within a different host, are disclosed in U.S. Pat. No. 5,639,641. Briefly, in a preferred method, (1) position alignments of a pool of antibody heavy and light chain variable regions is generated to give a set of heavy and light chain variable region framework surface exposed positions wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 angstroms (Å) of any atom of any residue of the complementarity-determining regions of the rodent antibody; and (5) the humanized rodent antibody having binding specificity is produced. Antibodies can be humanized using a variety of other techniques including CDR-grafting (EP 0239400; WO 91/09967; U.S. Pat. Nos. 5,530,101 and 5,585,089), veneering or resurfacing (EP 0592106; EP 0519596), and chain shuffling (U.S. Pat. No. 5,565,332). Human antibodies can be made by a variety of methods known in the art including phage display methods (U.S. Pat. Nos. 4,444,887, 4,716,111, 5,545,806, and 5,814,318).

Another technique which may be employed either as an alternative, or in addition, to the methods described above for reducing immunogenicity, is the “deimmunisation” of the antibody of the invention. Deimmunisation technology involves the identification and removal of T helper (Th) cell epitopes from antibody and other protein biological therapeutic agents. Th cell epitopes comprise short peptide sequences within proteins that have the capacity to bind to MHC class II molecules. The peptide-MHC class II complexes can be recognized by T cells and can trigger the activation and differentiation of Th cells, which is required to initiate and sustain immunogenicity through interaction with B cells, thus resulting in the secretion of antibodies that bind specifically to the administered biological therapeutic agent. For antibody deimmunisation, the Th-cell epitopes are identified within the antibody sequence, for example by a computer-based method for predicting the binding of peptides to human MHC class II molecules. To avoid recognition by T cells, the Th cell epitopes thus identified are eliminated from the protein sequence by amino acid substitutions. This may be achieved through the use of standard molecular biology techniques, such as for example site-directed mutagenesis to alter the nucleic acid sequence encoding the Th cell epitope in the therapeutic protein. In this way, an antibody or antigen-binding fragment may be modified so that HAMA (Human anti mouse antigenic) and/or anti-idiotypic response(s) are reduced or avoided. Thus, in specific embodiments, the antibodies of the invention may be modified to remove any Th cell epitopes present in their sequence. Such binding molecules are referred to herein as deimmunised antibodies.

The humanized antibodies of the invention preferably arise from the murine antibodies MPC5B4 described above. More preferably, they arise from the murine antibodies MPC5B4 disclosed in Field et al¹⁴.

Thus, in a specific embodiment, the present invention provides humanized antibodies or antigen-binding fragments thereof which specifically bind SEQ ID NO:1 and inhibit the interaction between human and mouse POSTN (any isoform) and their integrin ligands on epithelial cells, and subsequent signaling.

In another specific embodiment, the inhibitory antibody or antigen-binding fragment of the invention is fully human. The term “fully human” as used herein relates to an antibody or antigen-binding fragment whose amino acid sequences are derived from (i.e. originate or may be found in) humans.

Antibody Fragments of the Invention

The present invention can also be performed by using functional fragments of the inhibitory antibodies of the invention, as defined above. “Functional fragments” comprise a portion of an intact antibody, generally including the antigen binding or variable region of the intact antibody, here recognizing SEQ ID NO:1 in human and mouse POSTN protein. They preferably have the same inhibitory activity as the antibodies of the invention, as defined above.

In particular, functional fragments according to the invention can inhibit the binding of human and mouse POSTN to the αvβ3 integrins expressed by epithelial cells. This inhibitory activity can be assessed by evaluating the influence of the antibody fragment on the binding of human POSTN to αvβ3 and/or αvβ5-coated ELISA plates as disclosed in Field et al¹⁴. A functional fragment displays an inhibitory activity according to the invention, if its IC₅₀ for inhibiting human and mouse POSTN binding is below 30 μg/mL in the conditions of FIG. 2a of Field et al¹⁴. The inhibitory activity of said fragment can also be documented by studying its influence on the migration of endothelial cells, such as ECFC, in response to recombinant human or mouse POSTN or its Fas1-1 domain. As disclosed in Field et al¹⁴ (FIG. 5), the functional fragments of the invention are able to reduce POSTN-induced migration of ECFCs to the level of negative controls.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site.

Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “Fv” as used herein refers to the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three hyper variable regions specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. The antibody or fragment of the invention may also be multispecific, and in particular bispecific. As such, it can be chosen in the group consisting of: bispecific IgGs, IgG-scFv₂, (scFv)₄-IgG, (Fab′)₂, (scFv)₂, (dsFv)₂, Fab-scFv fusion proteins, (Fab-scFv)₂, (scFv)₂-Fab, (scFv-CH2-CH3-scFv)₂, bibody, tribody, bispecific diabody, disulfide-stabilized (ds) diabody, ‘knob-into whole’ diabody, single-chain diabody (scDb), tandem diabody (TandAb), flexibody, DiBi miniantibody, [(scFv)₂-Fc]₂, (scDb-CH3)₂, (scDb-Fc)₂, Di-diabody, Tandemab., etc.

As used therein, the term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, thereby resulting in a bivalent fragment, i.e., a fragment having two antigen-binding sites. Bispecific diabodies are heterodimers of two “cross-over” sFv fragments in which the VH and VL domains of the two antibodies are present on different polypeptide chains. Diabodies are described in greater detail in, for example, EP 404,097; WO 93/11161.

More particularly, the invention provides a functional fragment selected among the antibody fragments Fv, Fab, (Fab′)₂, Fab′, scFv, scFv-Fc and diabodies, or any fragment whose half-life has been increased by chemical modification.

The chemical modification as cited above, may be such as the addition of polyalkylene glycol such as polyethylene glycol (PEGylation) (PEGylated fragments are referred to as Fv-PEG, scFv-PEG, Fab-PEG, F(ab′)₂-PEG and Fab′-PEG), or by incorporation in a liposome, microspheres or Poly (D, L-lactic-co-glycolic acid) (PLGA), said fragments possessing at least six of CDRs of the invention which is notably capable of exerting in a general manner activity, even partial, of the antibody from which it arises.

Preferably, said antigen-binding fragment will comprise or include a partial sequence of the variable heavy or light chain of the antibody from which they are derived, said partial sequence being sufficient to retain the same binding specificity as the antibody from which it arises and sufficient affinity, preferably at least equal to 1/100, more preferably at least 1/10 of that of the antibody from which it arises.

Preferably, this antigen-binding fragment will be of the types Fv, scFv, Fab, F(ab′)₂, F(ab′), scFv-Fc or diabodies, which generally have the same binding specificity as the antibody from which they result.

According to the present invention, antigen-binding fragments of the invention can be obtained from the antibodies described above by methods such as enzyme digestion, including pepsin or papain, and/or by cleavage of the disulfide bridges by chemical reduction. The antigens-binding fragments can be also obtained by recombinant genetics techniques also known to a person skilled in the art or by peptide synthesis by means, for example, of automatic peptide synthesizers such as those sold by Applied BioSystems, etc.

Treatment Methods

As mentioned above, the antibodies or fragments of the present invention bind specifically to the epitope of SEQ ID NO:1 in the human and mouse POSTN protein, with a high affinity. They are thus capable of inhibiting POSTN-induced functions such as integrins-mediated intracellular signal transduction and inflammatory cell migration, as documented in ECFCs. POSTN is expressed by bronchial epithelial cells in response to Th2 cytokines IL-4 and IL-13 and represents a component of subepithelial fibrosis in bronchial asthma²².

Consequently, the antibodies and fragments of the invention are particularly useful for preventing or treating inflammatory and fibrotic respiratory disorders.

As used herein, the term “inflammatory and fibrotic respiratory disorders” refers to conditions or diseases resulting from the undesirable inflammation of the lung tissues. Such conditions include a number of chronic diseases, such as chronic obstructive pulmonary disease (COPD), idiopathic pulmonary fibrosis (IPF), and asthma. Other disorders comprise Acute respiratory distress syndrome (ARDS) or those resulting from infections.

More generally, inflammatory disorders which can be prevented, treated or managed in accordance with the methods of the invention include, but are not limited to, asthma, chronic obstructive pulmonary disease (COPD), Acute respiratory distress syndrome (ARDS), pulmonary fibrosis such as idiopathic pulmonary fibrosis (IPF), emphysema and lung inflammation or respiratory diseases resulting from viral, fungal or bacteria infections.

In a particular embodiment, the antibody of the invention or fragment thereof can be used to treat a respiratory disease induced by a viral infection, for example due to a betacoronavirus. A betacoronavirus infection in humans is usually diagnosed by a healthcare professional, based on observation of the infected patient's symptoms. Additional biological tests may be required to confirm the diagnosis: blood and/or sputum and/or bronchoalveolar fluid tests. Infection by a betacoronavirus can be established, for example, by molecular biological detection and/or viral titration of respiratory specimens, or by assaying blood for antibodies specific for said betacoronavirus. Conventional diagnostic methods comprise techniques of molecular biology such as PCR, which is well known to the person in the field.

In this particular embodiment, the antibody of the invention or fragment thereof can be used to treat a symptomatic COVID 19 disease. In the context of the invention, the term “COVID-19 disease” means the disease linked to the infection with the SARS-CoV-2 (Severe Acute Respiratory Syndrome Coronavirus 2). In the context of the invention, a “symptomatic COVID-19 disease” is characterized by a patient who shows at least one symptom of the COVID-19 disease. The most common symptoms of the COVID-19 disease are fever, muscle aches, headaches, fatigue, loss of taste and smell and respiratory symptoms such as a dry cough, difficulty breathing and a lack of oxygen. A symptomatic disease is in contrast to an asymptomatic disease which is characterized by a patient who is a carrier for a disease or infection but experiences no symptoms.

In another particular embodiment, the antibody of the invention or fragment thereof can be used to prevent the worsening of the mild COVID 19 disease into a “severe form”, i.e., into a COVID19 disease characterized by severe symptoms such as acute respiratory distress syndrome (ARDS) that requires hospitalization of the patient in any unit, and especially in the intensive care unit (ICU) in case of critical infection. In the context of the invention, the abbreviation “ICU” refers to an intensive care unit, a special department of a hospital or health care facility that provides intensive treatment medicine.

In this particular embodiment, the antibody or fragment of the invention is administered in priority to human patients that are older than 75 years old, or between 65 and 74 years old. These people indeed usually have greater mortality rates than younger people when infected with said virus.

As used herein, the terms “treat,” treating,” “treatment,” and the like refer to reducing or ameliorating the symptoms of a disorder (e.g., an inflammatory disorder) and/or symptoms associated therewith. It will be appreciated that, although not precluded, treating a disorder or condition does not require that the disorder, condition or symptoms associated therewith be completely eliminated.

As used herein “treating” a disease in a subject “in need thereof” or “treating” a subject “in need thereof” means subjecting the subject suffering from the said disorders to a pharmaceutical treatment, e.g., the administration of a drug, such that at least one symptom of the disease is decreased or prevented. As used herein, a treatment includes prophylactic administration (i.e., administration prior to the initiation of a pathologic event), and prevention of an attack or an exacerbation in ARDS or in chronic diseases such as asthma, or BPCO.

In one embodiment, “subject” refers to a mammal affected by a disorder characterized by inappropriate lung or respiratory inflammation. A “control subject” refers to a mammal wherein the lung/airways are not inflamed nor fibrotic. Said mammal includes human, dog, cat, cattle, goat, pig, swine, sheep and monkey. A human subject is herein designated as a “patient”.

In another embodiment, the present invention also relates to the use of an antibody or fragment of the invention for the preparation of a drug and/or a medicament and/or a pharmaceutical composition, for the prevention or the treatment of any of the above-mentioned diseases.

Said drug/medicament/pharmaceutical composition comprises the antibodies of the invention, or functional antigen-binding fragments thereof. Preferably, it contains, in addition to the antibody/fragments of the invention, various diluents, fillers, salts, buffers, stabilizers, solubilizers, and other materials well known in the art. More generally, it contains, in addition to the antibody/fragments of the invention, a pharmaceutically acceptable carrier.

As used herein, “pharmaceutically acceptable carrier” includes any and all solvents, buffers, salt solutions, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, topical, transdermal or oral administration. Pharmaceutically acceptable carriers include sterile aqueous solutions or dispersions and sterile powders for the extemporaneous preparation of sterile injectable solutions or dispersion. The use of media and agents for pharmaceutically active substances is well known in the art. A typical pharmaceutical composition for intravenous infusion could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17^{th} ed., Mack Publishing Company, Easton, Pa. (1985), and the 18^{th} and 19^{th} editions thereof, which are incorporated herein by reference. The antibody/fragment in the composition of the invention is preferably formulated in an effective amount. An “effective amount” refers to an amount which is effective, at dosages and for periods of time necessary, to achieve the desired result, such as prevention or treatment of respiratory diseases. A “therapeutically effective amount” means an amount which is sufficient to influence the therapeutic course of a particular disease state. A therapeutically effective amount is also one in which any toxic or detrimental effects of the agent are outweighed by the therapeutically beneficial effects.

According to another aspect, the invention relates to the use of the antibody of the invention, or antigen-binding fragments thereof, as a medicament. Also, the invention relates to the pharmaceutical composition of the invention, for use as a medicament.

The dosage of the antibodies in the compositions of the invention administered to a patient is typically about 0.1 mg/kg to about 10 mg/kg of the patient's body weight, e.g., about 0.1, about 0.2, about 0.3, about 0.4, about 0.5, about 0.6, about 0.7, about 0.8, about 0.9, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, and about 10 mg/kg of the patient's body weight. Preferably, the dosage of the antibodies administered to a patient is between about 1 mg/kg and about 9 mg/kg of the patient's body weight. In other embodiments the dosage of the antibodies in the compositions of the invention is about 0.1, about 0.3, about 1.0 or about 3.0 mg/kg of the patient's body weight.

The antibodies of the invention can be administered according to the judgment of the treating physician, e.g., daily, weekly, biweekly or at any other suitable interval, depending upon such factors, for example, as the nature of the ailment, the condition of the patient and half-life of the antibody. In a preferred example, a subject is treated with the antibody or fragment of the invention in the range of between about 0.1 to about 10 mg/kg body weight, one time per 1-3 weeks for between about 1 to about 10 weeks, preferably between about 2 to about 8 weeks, more preferably between about 3 to about 7 weeks, and even more preferably for about 4, about 5, or about 6 weeks. In other embodiments, the pharmaceutical compositions of the invention are administered once a day, twice a day, or three times a day. In other embodiments, the pharmaceutical compositions are administered once a week, twice a week, once every two-three weeks, once a month, once every six weeks, once every two months, twice a year or once per year. It will also be appreciated that the effective dosage of the antibodies used for treatment may increase or decrease over the course of a particular treatment.

In a most preferred embodiment, the composition of the invention is administered intravenously over about 30 minutes. In other embodiments, the composition of the invention is administered intravenously over at least about 1 hour, at least about 30 minutes, or at least about 15 minutes.

More generally, for therapeutic applications, the antibody of the invention could be administered to the subject as a bolus or by continuous infusion over a period of time, by intramuscular, intraperitoneal, intracerebrospinal, subcutaneous, intraarticular, intrasynovial, intrathecal, oral, topical, or inhalation routes.

In a most preferred embodiment, the composition of the invention is administered by inhalation, with a spray.

### FIGURE LEGENDS

FIG. 1 shows that papain increases POSTN production in mice. (A) To assess the capacity of papain to induce POSTN production in asthma allergic, mice were treated or not with 25 μg papain intratracheally (i.t.). Mice were euthanized at 48 h post papain-administration. B) POSTN levels was quantify by ELISA in BALF samples. Results are expressed as mean±SEM (n=5). Statistical analysis on day 2 was determined using the Mann-Whitney test. *p<0.05 compared to control-mice

FIG. 2 describes that the blockage of POSTN prevents lung inflammation in asthma. A) To assess the capacity of anti-POSTN antibody treatment to prevent asthma, mice were injected with the anti-POSTN MPC5B4 antibody or Isotype control antibody (12.5, 5, 2.5 mg/kg; i.p.). 1 h after injection mice were treated or not with 25 μg papain intranasal for 3 days to induce asthma severe. Mice were euthanized at day 4. B) Doses response study of monoclonal anti-POSTN antibody in asthma allergic. Eosinophils and lymphocytes number in BALF. Results are expressed as mean±SEM (n>5). Statistical analysis on day 4 was determined using the one-way Anova test multiple comparisons. * p<0.05 compared to control untreated-mice. #p<0.05 compared to papain-treated-mice.

FIG. 3 discloses that the systemic blockage of POSTN prevents lung inflammation in asthma. A) To assess the capacity of anti-POSTN antibody systemic treatment to prevent asthma, mice were injected intra-peritoneally with the anti-POSTN MPC5B4 or Isotype control antibody (12.5 mg/kg; i.p.). 1 h after injection, mice were treated or not with 25 μg papain intranasally for 3 days to induce severe asthma. Mice were euthanized at day 4. B) Total cell, C-D) Eosinophils, Lymphocytes and Monocytes infiltration was monitored in BALF at day 4 post-administration. D) Eosinophils in lung and CCL17 production was quantified in lung by ELISA at day 4. E) Tissue damage of lung on day 4 after 3 administrations of papain with or without the anti-POSTN antibody. Results are expressed as mean±SEM (n=5). Statistical analysis on day 4 was determined using the one-way Anova test multiple comparisons. *p<0.05 compared to control untreated-mice. #p<0.05 compared to papain-treated-mice.

FIG. 4 shows that the blockage of POSTN locally reduces lung inflammation in severe asthma. A) To assess the capacity of anti-POSTN antibody local treatment to prevent asthma, mice were injected intra-tracheally with the anti-POSTN MPC5B4 (10, 1 mg/kg; i.t.). 1 h after injection mice were treated or not with 25 μg papain for 3 days to induce asthma. Mice were euthanized at day 4. B) Total cells, C) Macrophages, Eosinophils, Lymphocytes, and Neutrophils infiltration was monitored in BALF at day 4 post-administration. D) POSTN and MPO production was quantified in BALF by ELISA. Results are expressed as mean±SEM (n>3). Statistical analysis on day 4 was determined using the one-way Anova test multiple comparisons. *p<0.05 compared to control untreated-mice. #p<0.05 compared to papain-treated-mice.

FIG. 5 shows that the inhibition of POSTN reduces lung inflammation and collagen deposition in a severe asthma model. A) To assess the capacity of anti-POSTN antibody treatment to treat asthma, mice were treated with saline vehicle or with 4 administrations of papain (25 μg i.n.) on day 1, 2, 14 and 21 to induce asthma, with or without anti-POSTN antibody MPC5B4 (12.5 mg/kg; i.p.) on day 14, 18 and 21. Mice were euthanized at day 22. B) Total cells, Eosinophil, Lymphocyte, Macrophage and Neutrophil infiltration was monitored in BALF on day 22. C) Tissue damage, soluble collagen protein and collagen message transcription in lung were monitored on day 22. Results are expressed as mean±SEM (n=3). *p<0.05 compared to control isotype treated-mice.

FIG. 6 shows that the treatment with the anti-POSTN antibody MPC5B4 prevents lung inflammation in a pulmonary fibrosis model. A) To assess the capacity of anti-POSTN antibody prophylactic treatment to prevent fibrosis, mice received three doses of the monoclonal anti-POSTN MPC5B4 or Isotype control antibody (1.25 mg/kg; i.p.) on day 0, 5 and 10. 1 h after the first antibody injection, mice were treated or not with bleomycin (BLM, 3 mg/kg i.n.) to induce pulmonary fibrosis. Mice were euthanized at day 14. B) To analyze the therapeutic efficacy of the monoclonal anti-POSTN antibody, two doses of MPC5B4 were administrated by intraperitoneal route on day 7 and 10 after BLM administration as in A. C) Total cells, neutrophils and lymphocytes infiltration was monitored in BALF at day 14 post-BLM administration.

FIG. 7 shows that the LPS increases POSTN production in mice. A) To assess the capacity of LPS to induce POSTN production, mice were treated or not with LPS (10 μg i.t.). Mice were euthanized at 48 h post LPS-administration. B) POSTN levels were quantified by ELISA in BALF samples. Results are expressed as mean±SEM (n=3-4). Statistical analysis on day 2 was determined using the Mann-Whitney test. *p<0.05 compared to control untreated-mice.

FIG. 8 discloses that the blockage of POSTN prevents lung inflammation due to infections. A) To assess the capacity of anti-POSTN antibody treatment to prevent exacerbations, mice were injected with MPC5B4 or with an anti-GM-CSF antibody (12.5 mg/kg; i.p.). 1 h after injection mice were treated or not with LPS (1 μg intranasally). Mice were euthanized at 24 h post-LPS administration. B) Total cells, neutrophils and lymphocytes infiltration was monitored in BALF at 24 h post-LPS administration. Results are expressed as mean±SEM (n>5). Statistical analysis was determined using the one-way Anova test multiple comparisons. *p<0.05 compared to control untreated mice. #p<0.05 compared to LPS-treated-mice.

### EXAMPLES

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

I. Material and Methods

Mice

Male eight-weeks old C5BL/6 mice were purchased from Janvier Labs (Saint Berthevin, France). Mice were maintained under a 12-hours light-dark cycle and they were fed with a standard laboratory diet. All studies were approved by Institutional Animal Care and Use Committee of CNRS.

Papain-Induced Lung Inflammation Model in Mice

Mice were anesthetized by 2-3% isofluorane and 2% O₂ follow asthma was induced by the administration of papain (Calbiochem, Darmstadt, Germany). Papain was dissolved in sterile NaCl solution. Animal were sensitized 3 times with 40 ul solution containing 25 μg papain administrated intra-nasally or intra-tracheal. Mice were sacrificed at day 4 following asthma induction. Control animals were either untreated.

Mice were euthanized by CO₂ inhalation 4 days after papain administration and BALF was collected. After a hearth perfusion with ISOTON II (acid-free balanced electrolyte solution Beckman Coulter, Krefeld, Germany), lungs were collected and sampled for analyses.

Bleomycin-Induced Lung Fibrosis Model in Mice

Mice were anesthetized by 2-3% isofluorane and 2% O₂ follow pulmonary fibrosis was induced by the administration of bleomycin (Merck KGaA, Darmstadt, Germany). Bleomycin was dissolved in sterile NaCl solution. Animal were sensitized 3 times with 40 μl solution containing 3 mg/kg administrated intranasally. Mice were sacrificed at day 14 following emphysema induction. Control animals were either untreated. BALF and lungs were collected and sampled for analyses.

LPS-Induced Lung Infection Model in Mice

Mice were anesthetized by 2-3% isofluorane and 2% O₂ follow asthma was induced by the administration of LPS. LPS was dissolved in sterile NaCl solution. Animal were sensitized 3 times with 40 μl solution containing 1 μg LPS administrated intra-nasally or intra-tracheal. Mice were sacrificed 24 h following LPS administration. Control animals were either untreated. Mice were euthanized by CO₂ inhalation and BALF was collected. After a hearth perfusion with ISOTON II, lungs were collected and sampled for analyses.

Antibody Administration

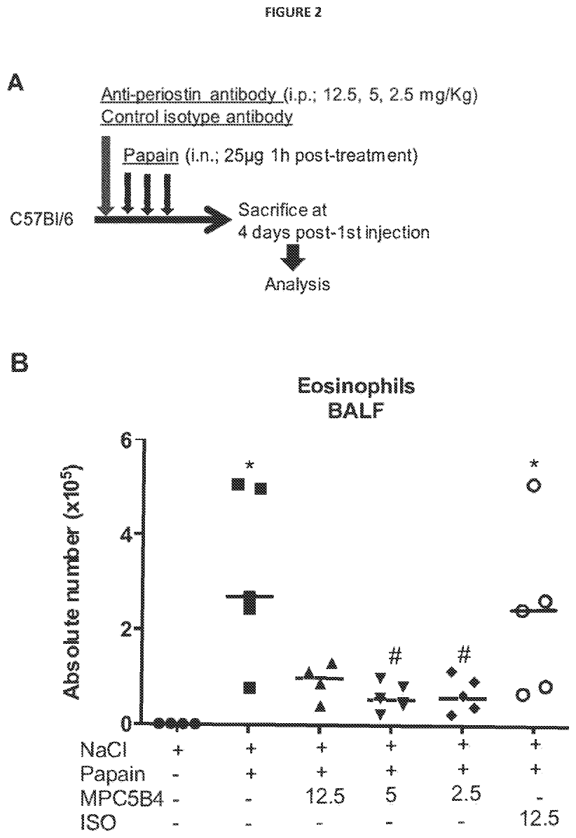

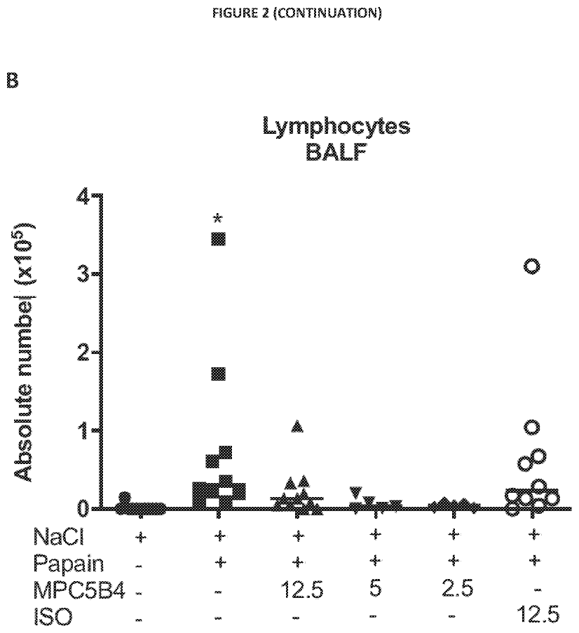

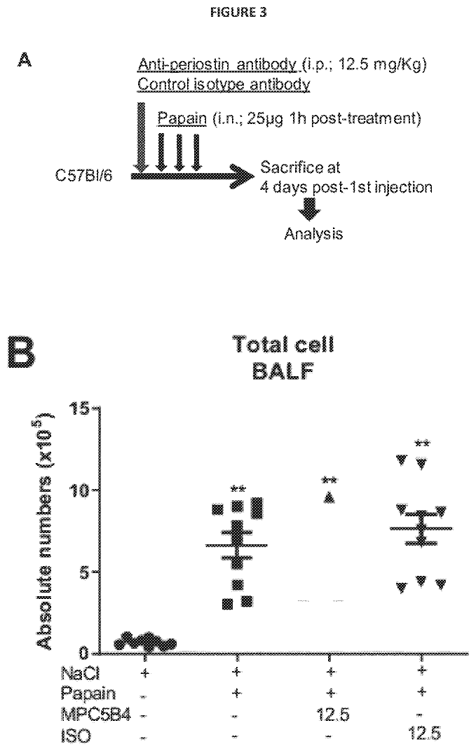

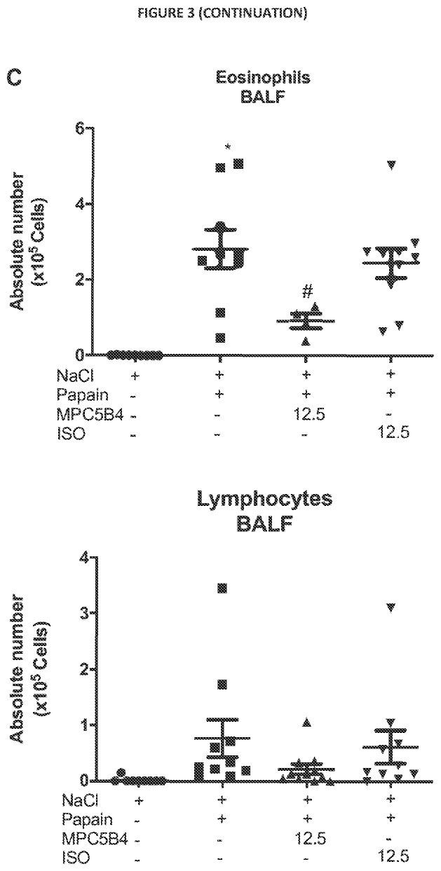

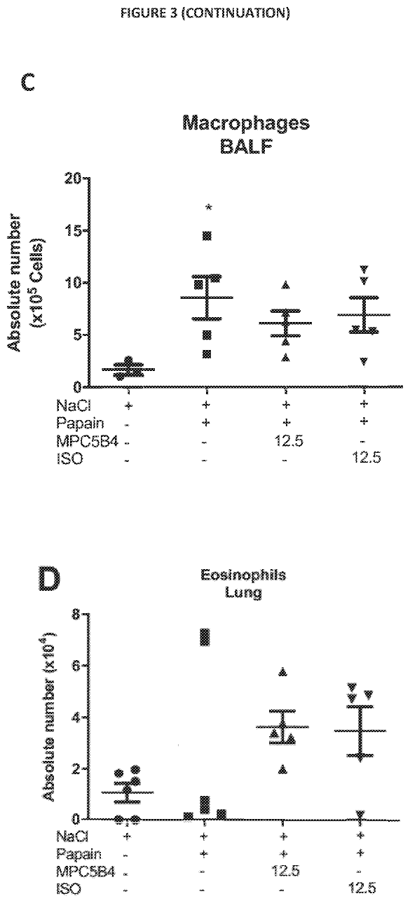

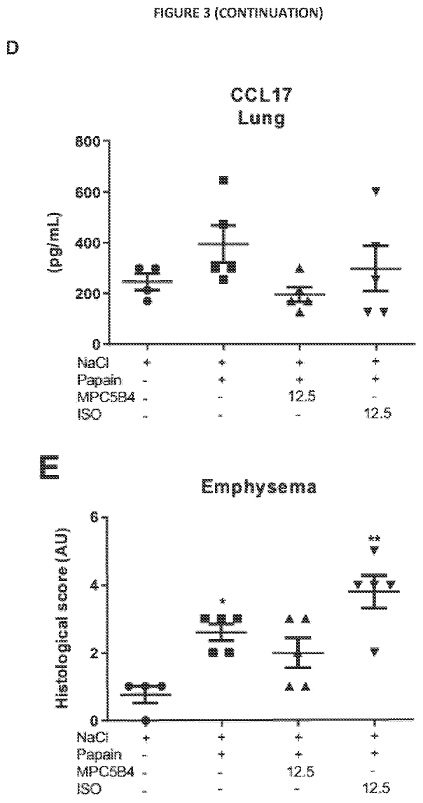

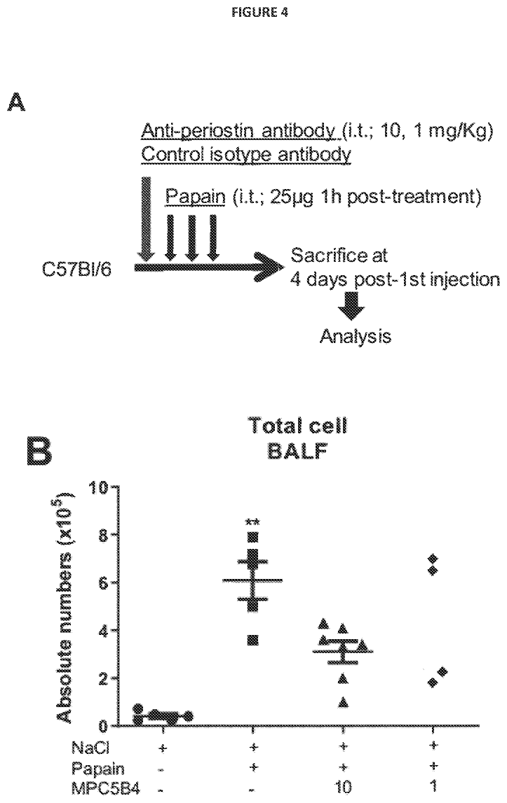

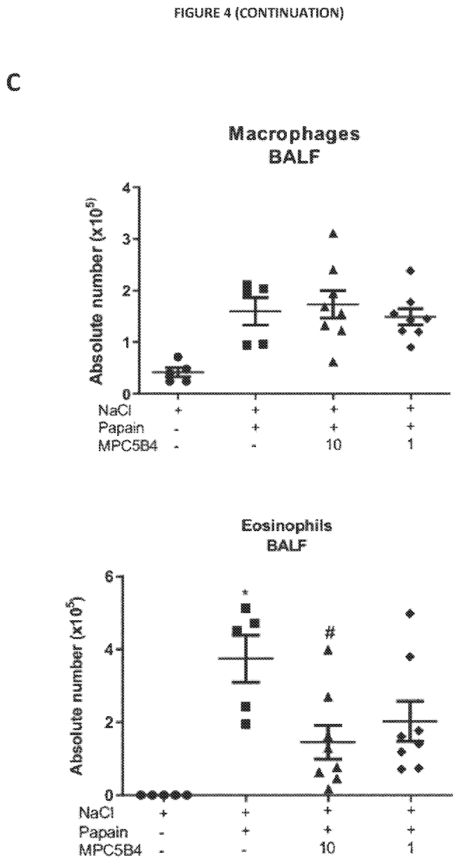

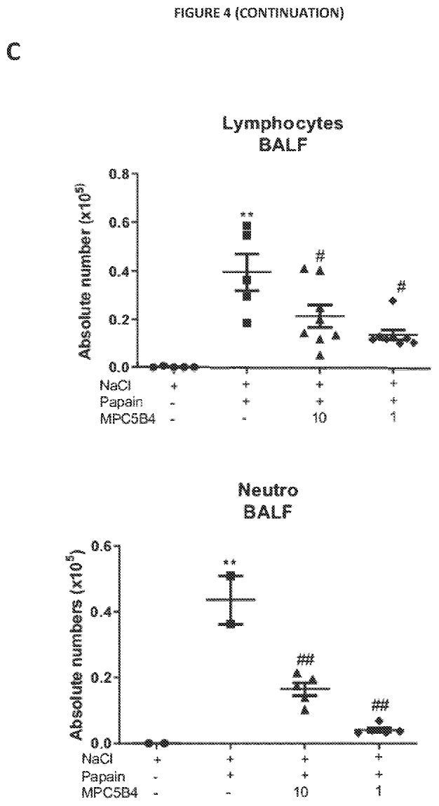

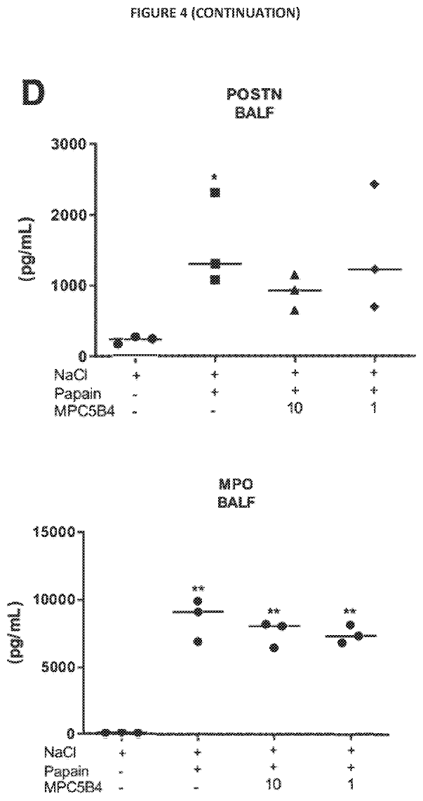

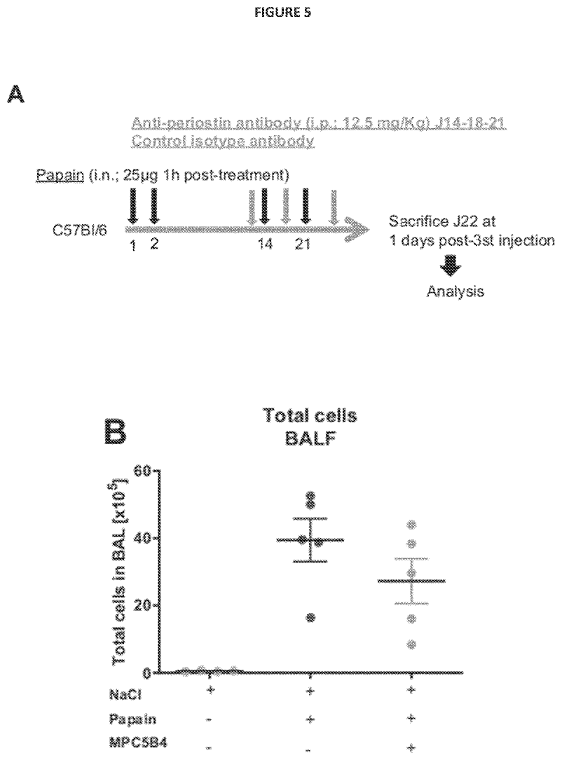

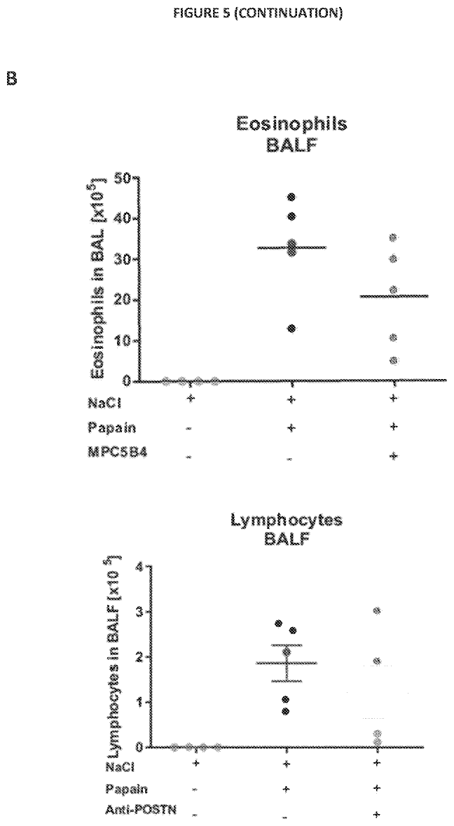

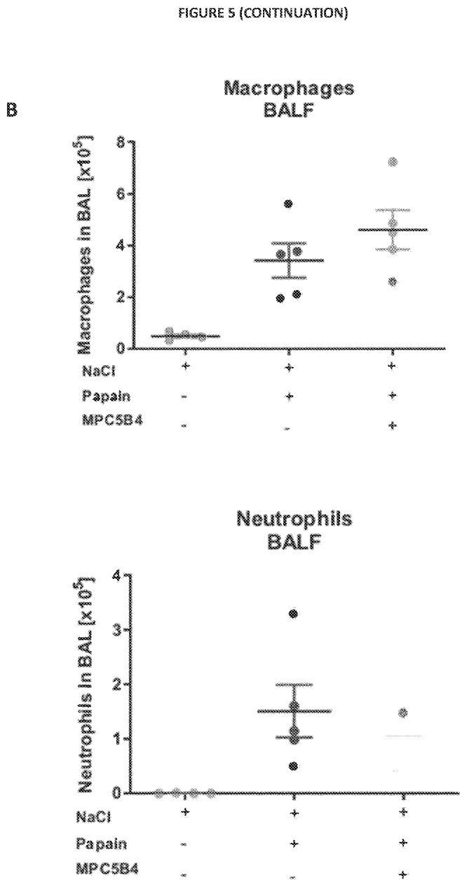

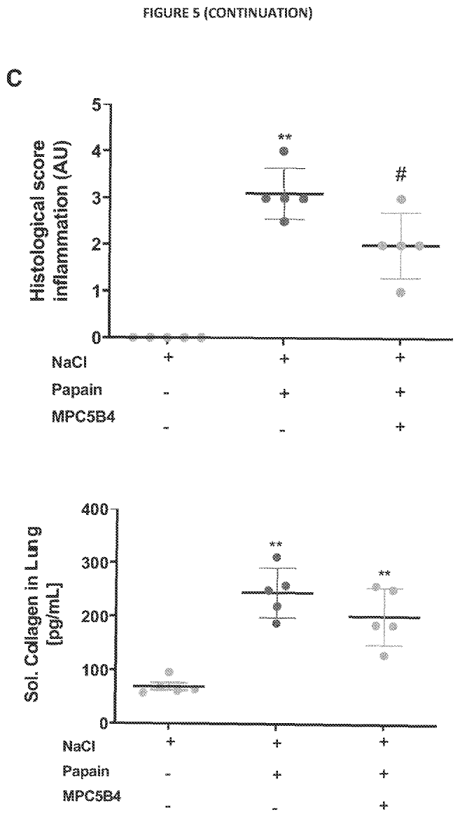

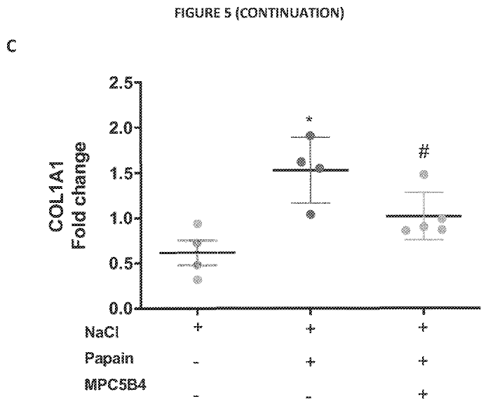

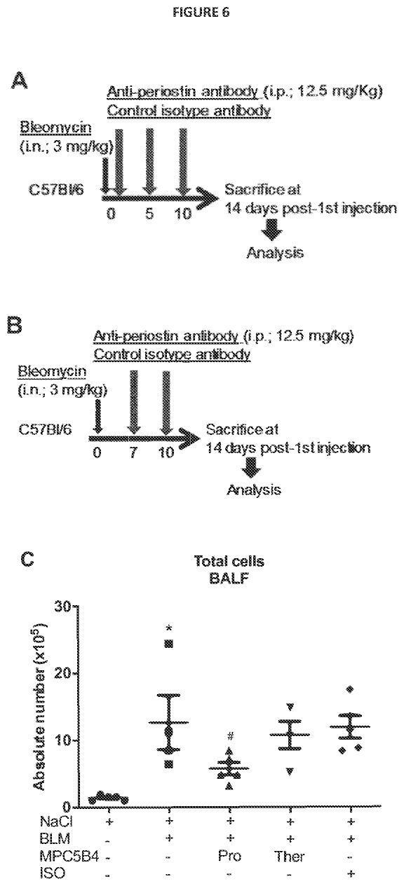

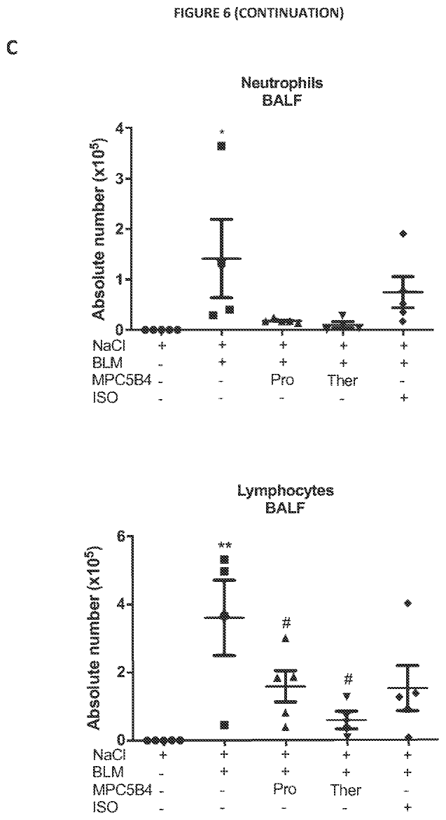

Anti-POSTN (MPC5B4) or isotype control (IgG1) antibody were given at indicated time points (FIG. 2A, FIG. 3A, FIG. 4A, FIG. 5A-B and FIG. 6A). Different doses of monoclonal antibody (12.5, 5, 2.5 mg/kg; i.p. or 10, 3, 1 mg/kg; it.) were used for dose response study.

Sample Size and Statistical Analysis

The statistical analysis was performed using Prism 5 (GraphPad software, San Diego, CA). Results were analyzed using non-parametric test (Mann Whitney tests), T test expressed in terms of probability (P). Differences were considered significant when p<0.05. All data were expressed as mean±SEM.

II. Results

Papain Increases POSTN Production.

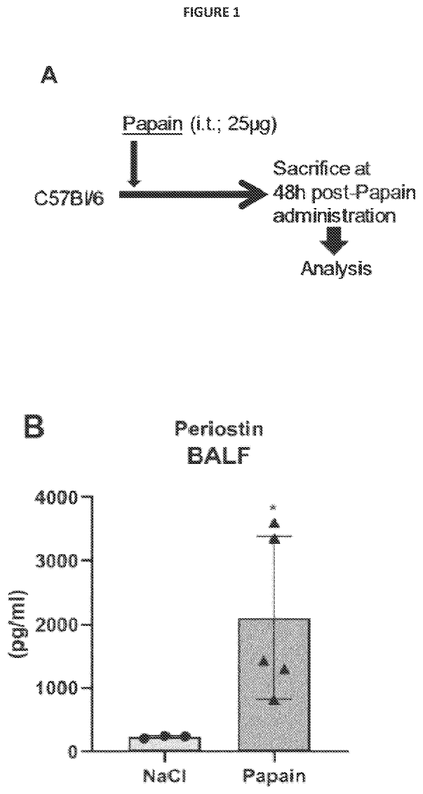

To assess the capacity of papain to induce periostin (POSTN) production, C57BL/6 mice were treated or not with 25 μg papain intratracheally. Mice received 1 dose of papain (FIG. 1A). POSTN levels were measure 48 h after papain administration. Papain-treated mice show significant increased levels of POSTN in bronchoalveolar lavage fluid (BALF) compared to control mice (FIG. 1B).

Anti-POSTN Antibody Prevents Lung Inflammation in Asthma.

Monoclonal mouse anti-POSTN antibody (MPC5B4, IgG1 isotype) blocks POSTN binding site interaction (aa140-150) with integrin αvβ3¹⁴. The treatment with MPC5B4 was included to assess the impact of POSTN signaling modulation in lung inflammation. C57BL/6 mice were injected with anti-POSTN or Isotype control antibody (12.5, 5, 2.5 mg/kg; i.p.). 1 h after injection, mice were treated or not with papain (25 μg i.n.) to induce allergic asthma. Mice received 3 doses of papain on 3 consecutive days (FIG. 2A). Cell count was monitored in bronchoalveolar lavage fluid (BALF) and lung 4 days after first administration. A dose response analysis shows the efficiency of anti-POSTN antibody therapy to control lung inflammation in asthma allergic (FIG. 2B). Papain treated mice as control group shown increase total cells count, eosinophils and lymphocyte number (FIG. 2B). Moreover, papain treated mice show that anti-POSTN therapy protected the recipient mice against lung inflammation reducing significantly eosinophils and lymphocytes counts in BALF (FIG. 2B). However, control isotype antibody therapy failed to protect the recipient mice against inflammation.

After the dose response study, C57BL/6 mice were injected with anti-POSTN or Isotype control antibody (12.5 mg/kg; i.p.). 1 h after injection, mice were treated or not with 25 μg papain to induce allergic asthma. Mice received 3 doses of papain on 3 consecutive days (FIG. 3A). Cell counts were monitored in BALF and lung 4 days after first administration. Papain treated mice as control group showed an increase in total cells count (FIG. 3B), eosinophils, lymphocyte and monocytes number (FIG. 3C-D). Moreover, papain treated mice showed that anti-POSTN therapy protected the recipient mice against lung inflammation reducing significantly eosinophils and lymphocytes counts (FIG. 2B-C). However, control isotype antibody therapy failed to protect the recipient mice against inflammation. Thymus- and activation-regulated chemokine (TARC/CCL17) induces a Th2-dominated inflammatory reaction in mice. Papain treated mice showed an increase in CCL17 levels in lung, however the anti-POSTN antibody treatment reduced the CCL17 production (FIG. 3D). On the other hand, papain treated mice significantly increased the tissue damage in lung measured by histological score emphysema after the 4 days. Anti-POSTN antibody treatment reduced the lung tissue damage (FIG. 3E).

Local Administration of Anti-POSTN Antibody Prevents Lung Inflammation.

The treatment local with the anti-POSTN antibody MPC5B4 by intratracheal route was included also to assess the impact of POSTN in lung inflammation. C57BL/6 mice were injected with the anti-POSTN antibody MPC5B4 (10, 1 mg/kg; i.t.). 1 h after injection, mice were treated or not with 25 μg papain to induce asthma allergic. Mice received 3 doses of papain (FIG. 4A). Cells counts were monitored in BALF 4 days after the first administration. Papain treated mice as control group showed an increase of total cells count, eosinophils, lymphocyte, neutrophils and macrophages number (FIG. 4B,C). Moreover, papain treated mice showed that anti-POSTN therapy protected the recipient mice against lung inflammation reducing significantly eosinophils, lymphocytes and neutrophils counts (FIG. 4B,C).

Papain treated mice showed an increase in POSTN and myeloperoxidase (MPO) levels in lung, however, the anti-POSTN antibody treatment reduced the POSTN production (FIG. 4D).

Anti-POSTN Antibody Blocks Chronic Severe Papain Induced Asthma

To assess the capacity of anti-POSTN antibody to treat chronic severe asthma, mice were treated with saline vehicle or with 4 administrations of papain (25 μg i.n.) on day 1, 2, and further on days 14 and 21 (FIG. 5A). Anti-POSTN antibody MPC5B4 (12.5 mg/kg; i.p.) administered on day 14, 18 and 21 reduced lung inflammation and collagen deposition in this severe asthma model. Total cells, Eosinophil, Lymphocyte, Macrophage and Neutrophil infiltration were reduced in BALF on day 22 (FIG. 5B), as were lung tissue damage, soluble collagen protein and collagen message transcription (FIG. 5C) after anti-POSTN MPC5B4 antibody as compared to isotype control treated-mice.

Anti-POSTN Antibody Reduces Pulmonary Fibrosis.

The treatment with monoclonal anti-POSTN antibody (MPC5B4) was also included to assess the impact of POSTN signaling modulation in pulmonary fibrosis. C57BL/6 mice were injected with anti-POSTN or Isotype control antibody (12.5 mg/kg; i.p.). Mice received three doses of monoclonal antibody as prophylactic treatment (FIG. 6A). 1 h after the first injection, mice were treated or not with bleomycin (3 mg/kg, i.n.) to induce lung fibrosis. Cell counts were monitored in BALF and lung 14 days after first administration. Bleomycin-treated mice as control group showed an increase in total cells, neutrophils and lymphocyte numbers (FIG. 6B and 6C). Moreover, bleomycin-treated mice showed that prophylactic anti-POSTN (Pro) protected the recipient mice against lung fibrosis, reducing significantly neutrophils and lymphocytes numbers (FIG. 6C). However, control isotype antibody failed to protect the recipient mice against fibrosis (FIG. 6C).

On the other hand, monoclonal antibody was administrated twice to show its therapeutic efficacy (FIG. 6B). Bleomycin-treated mice showed that therapeutic monoclonal anti-POSTN (MPC5B4) treatment (Ther) significantly reduced the lung inflammation measured by inflammatory cells count in the BALF after the 14 days (FIG. 6C). Histopathological analysis of the lung showed that MPC5B4 treatment in bleomycin-treated mice significantly ameliorates inflammation and tissue remodeling, compared to isotype control antibody treated mice (not shown). Statistical analysis on day 14 was determined using the one-way Anova test multiple comparisons. *p<0.05 compared to control untreated mice. #p<0.05 compared to bleomycin-treated-mice.

LPS Increases POSTN Production.

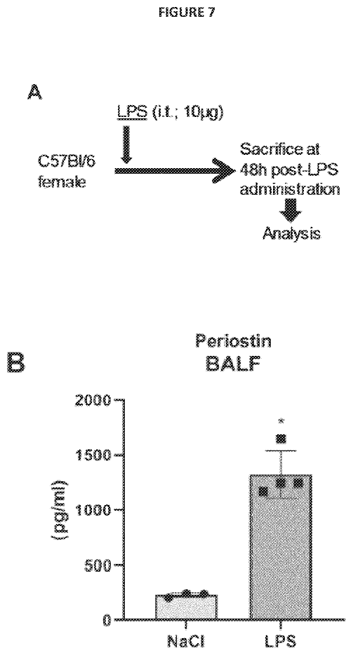

To assess the capacity of LPS to induce periostin production, C57BL/6 mice were treated with 10 μg LPS or saline vehicle intratracheally and POSTN levels were measured 48 h after. LPS-treated mice showed significantly increased levels of POSTN in BALF, compared to control mice (FIG. 7B).

Anti-POSTN Antibody Prevents Lung Inflammation in Exacerbation.

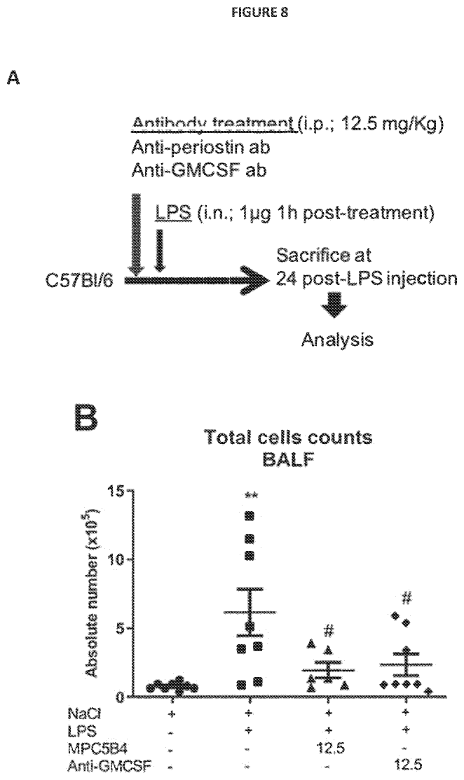

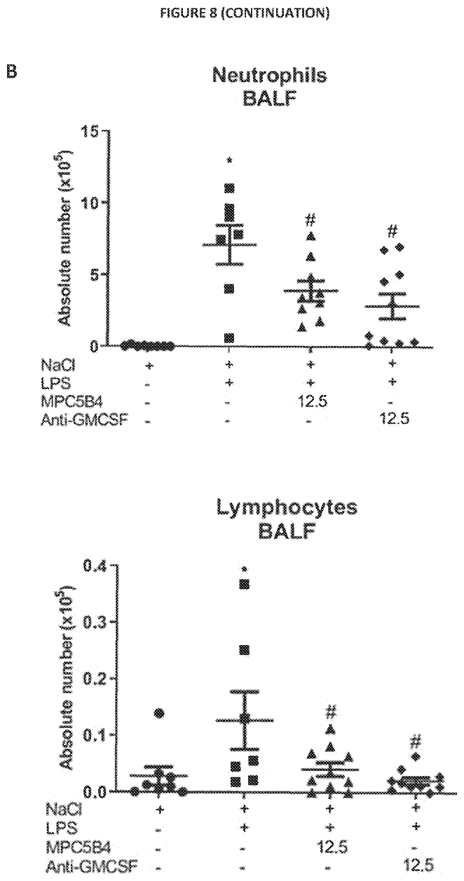

The treatment with MPC5B4 was included to assess the impact of POSTN signaling modulation in exacerbation of lung diseases. C57BL/6 mice were injected with the anti-POSTN MPC5B4 or with an anti-GMCSF antibody (12.5 mg/kg; i.p.). 1 h after injection, mice were treated or not with 1 μg LPS to induce inflammation. Mice received 1 dose of LPS (FIG. 8A). Cell counts were monitored in BALF, at 24 h. LPS-treated mice as control group showed an increase in the total cell count, neutrophils and lymphocyte numbers (FIG. 8B). Moreover, the anti-POSTN therapy protected the recipient mice against lung inflammation reducing significantly neutrophils and lymphocytes counts in BALF (FIG. 8B), to a level similar to an anti-GM-CSF antibody used as a reference compound for blocking LPS-induced inflammation.

### REFERENCES

1. Aghasafari P, George U, Pidaparti R. A review of inflammatory mechanism in airway diseases. Inflamm Res 2019; 68:59-74.
2. Durham A L, Caramori G, Chung K F, Adcock I M. Targeted anti-inflammatory therapeutics in asthma and chronic obstructive lung disease. Transl Res 2016; 167:192-203.
3. Diamond M, Peniston Feliciano H L, Sanghavi D, Mahapatra S. Acute Respiratory Distress Syndrome. In: StatPearls. Treasure Island (FL); 2020.
4. Hansbro P M, Kaiko G E, Foster P S. Cytokine/anti-cytokine therapy—novel treatments for asthma? Br J Pharmacol 2011; 163:81-95.
5. Nanri Y, Nunomura S, Terasaki Y, Yoshihara T, Hirano Y, Yokosaki Y, et al. Cross-Talk between Transforming Growth Factor-beta and Periostin Can Be Targeted for Pulmonary Fibrosis. Am J Respir Cell Mol Biol 2020; 62:204-16.
6. Bozyk P D, Bentley J K, Popova A P, Anyanwu A C, Linn M D, Goldsmith A M, et al. Neonatal periostin knockout mice are protected from hyperoxia-induced alveolar simplication. PLoS One 2012; 7:e31336.
7. Izuhara K, Conway S J, Moore B B, Matsumoto H, Holweg C T, Matthews J G, et al. Roles of Periostin in Respiratory Disorders. Am J Respir Crit Care Med 2016; 193:949-56.
8. Kudo A. Periostin in fibrillogenesis for tissue regeneration: periostin actions inside and outside the cell. Cell Mol Life Sci 2011; 68:3201-7.
9. Liu A Y, Zheng H, Ouyang G. Periostin, a multifunctional matricellular protein in inflammatory and tumor microenvironments. Matrix Biol 2014; 37:150-6.
10. Horiuchi K, Amizuka N, Takeshita S, Takamatsu H, Katsuura M, Ozawa H, et al. Identification and characterization of a novel protein, periostin, with restricted expression to periosteum and periodontal ligament and increased expression by transforming growth factor beta. J Bone Miner Res 1999; 14:1239-49.
11. Morra L, Rechsteiner M, Casagrande S, von Teichman A, Schraml P, Moch H, et al. Characterization of periostin isoform pattern in non-small cell lung cancer. Lung Cancer 2012; 76:183-90.
12. Morra L, Moch H. Periostin expression and epithelial-mesenchymal transition in cancer: a review and an update. Virchows Arch 2011; 459:465-75.
13. Ruan K, Bao S, Ouyang G. The multifaceted role of periostin in tumorigenesis. Cell Mol Life Sci 2009; 66:2219-30.
14. Field S, Uyttenhove C, Stroobant V, Cheou P, Donckers D, Coutelier J P, et al. Novel highly specific anti-periostin antibodies uncover the functional importance of the fasciclin 1-1 domain and highlight preferential expression of periostin in aggressive breast cancer. Int J Cancer 2016; 138:1959-70.
15. Orecchia P, Conte R, Balza E, Castellani P, Borsi L, Zardi L, et al. Identification of a novel cell binding site of periostin involved in tumour growth. Eur J Cancer 2011; 47:2221-9.
16. Uyttenhove C, Marillier R G, Tacchini-Cottier F, Charmoy M, Caspi R R, Damsker J M, et al. Amine-reactive OVA multimers for auto-vaccination against cytokines and other mediators: perspectives illustrated for GCP-2 in L. major infection. J Leukoc Biol 2011; 89:1001-7.
17. Gomez K A, Coutelier J P, Retegui L A. Changes in the specificity of antibodies in mice infected with lactate dehydrogenase-elevating virus. Scand J Immunol 1997; 46:168-74.
18. Dall'Acqua W F, Woods R M, Ward E S, Palaszynski S R, Patel N K, Brewah Y A, et al. Increasing the affinity of a human IgG1 for the neonatal Fc receptor: biological consequences. J Immunol 2002; 169:5171-80.
19. Zalevsky J, Chamberlain A K, Horton H M, Karki S, Leung I W, Sproule T J, et al. Enhanced antibody half-life improves in vivo activity. Nat Biotechnol 2010; 28:157-9.
20. Lee C H, Kang T H, Godon O, Watanabe M, Delidakis G, Gillis C M, et al. An engineered human Fc domain that behaves like a pH-toggle switch for ultra-long circulation persistence. Nat Commun 2019; 10:5031.
21. Shields R L, Namenuk A K, Hong K, Meng Y G, Rae J, Briggs J, et al. High resolution mapping of the binding site on human IgG1 for Fc gamma RI, Fc gamma RII, Fc gamma RII, and FcRn and design of IgG1 variants with improved binding to the Fc gamma R. J Biol Chem 2001; 276:6591-604.
22. Takayama G, Arima K, Kanaji T, Toda S, Tanaka H, Shoji S, et al. Periostin: a novel component of subepithelial fibrosis of bronchial asthma downstream of IL-4 and IL-13 signals. J Allergy Clin Immunol 2006; 118:98-104.
23. Naik P K. et al, Periostin promotes fibrosis and predicts progression in patients with idiopathic pulmonary fibrosis, Am. J. Physiol. Lung Cell Mol. Physiol. 303: L1046-1056, 2012
24. Bentley J. K., et al. Periostin is required for maximal airways inflammation and hyperresponsiveness in mice. J. Allergy Clin. Immunol. 2014

1. A monoclonal anti-periostin antibody that recognizes specifically and with high affinity the peptide sequence SEQ ID NO:1 in the fasciclin (FAS)1-1 domain of periostin, or a functional antigen-binding fragment thereof, for use as a medicament.

2. The monoclonal anti-periostin antibody of claim 1, for use for treating inflammatory, fibrotic or respiratory disorders in a subject in need thereof.

3. The monoclonal anti-periostin antibody for use according to claim 2, wherein said disorders are chosen in the group consisting of: asthma, Acute respiratory distress syndrome (ARDS), chronic obstructive pulmonary disease (COPD), pulmonary fibrosis such as idiopathic pulmonary fibrosis (IPF), emphysema and lung inflammation or respiratory diseases resulting from viral, fungal or bacteria infections.

4. The monoclonal anti-periostin antibody for use according to claim 3, wherein said disorder is a symptomatic form of the COVID 19 disease.

5. The monoclonal anti-periostin antibody for use according to claim 1, wherein it is an IgG, preferably an IgG1.

6. The monoclonal anti-periostin antibody for use according to claim 1, wherein it inhibits the binding of human POSTN to αvβ3 integrins with an IC₅₀ below 30 μg/mL.

7. The monoclonal anti-periostin antibody for use according to claim 1, wherein it has a dissociation constant (K_{D}) with SEQ ID NO:1 of about 0.08.

8. The monoclonal anti-periostin antibody for use according to claim 1, wherein it has been produced by immunizing a mouse with SEQ ID NO:1 or POSTN conjugated to ovalbumine, said mouse having been previously infected with a lactate dehydrogenase-elevating virus.

9. The monoclonal anti-periostin antibody for use according to claim 1, wherein it is a chimeric, a humanized or a full-human antibody.

10. The monoclonal anti-periostin antibody for use according to claim 1, wherein it has been recombinantly modified by introducing a N297A mutation in the human IgG1 heavy chain.

11. The monoclonal anti-periostin antibody for use according to claim 1, wherein said functional fragment is a Fab, Fab′, F(ab′)₂ or Fv fragment having the same dissociation constant as the antibody of claim 7.
